(19) United States
(12) Patent Application Publication
(10) Pub. No.: US 2024/0180869 A1
(43) Pub. Date: Jun. 6, 2024

(54) TREATMENT OF MULTIPLE SCLEROSIS USING PKC ACTIVATORS

(71) Applicant: SYNAPTOGENIX, INC., New York, NY (US)

(72) Inventor: Daniel L. ALKON, Chevy Chase, MD (US)

(73) Assignee: SYNAPTOGENIX, INC., New York, NY (US)

(21) Appl. No.: 18/547,266

(22) PCT Filed: Feb. 8, 2022
(86) PCT No.: PCT/US2022/015585

§ 371 Date: Aug. 21, 2023

Related U.S. Application Data
(60) Provisional application No. 63/146,801, filed on Feb. 8, 2021.
(60) Provisional application No. 63/168,465, filed on Mar. 31, 2021.

(51) Int. Cl. A61K 31/366; A61K 9/00; A61K 31/215

(52) U.S. Cl. CPC A61K 31/366; A61K 9/0019; A61K 31/215

(57) ABSTRACT

A method for treating or preventing mild, moderate, or advanced multiple sclerosis (MS) in a subject, the method comprising administering to the subject a PKC activating compound (e.g., a bryostatin, such as bryostatin-1, or a bryolog) in a therapeutically effective amount to result in mitigation or prevention of demyelination, initiation of remyelination, or reduction or prevention of neuroinflammation in early MS. The MS may be, for example, relapsing-remitting MS (RRMS), primary-progressive MS (PPMS), secondary-progressive MS (SPMS), or progressive-relapsing MS (PRMS). The PKC activating compound may be administered at an initial loading dose of about 15, 24, or 48 micrograms weekly in the first one week or consecutive two or three weeks, followed by doses of about 12, 20, or 40 micrograms alternately every two or three weeks for at least 4, 5, 6, 8, 10, 12, 14, 16, 18, 20, 24, or 30 total weeks.

### BACKGROUND

Multiple sclerosis (MS) is a debilitating neurological disorder characterized by the demyelination of nerve cells in the brain and spinal cord. The following four types of MS have been identified: relapsing-remitting MS (RRMS), primary-progressive MS (PPMS), secondary-progressive MS (SPMS), and progressive-relapsing MS (PRMS). Some of the early symptoms of the disease may include vision problems associated with inflammation of the optic nerve (i.e., optic neuritis), fatigue, cognitive decline, spasms, sensory impairment, dysphagia, and incontinence. In some subjects, MS may present mild or manageable symptoms for the entire duration of the disease. However, in a minority of subjects, MS may present more advanced symptoms, such as vision loss, substantial loss of mobility, substantial pain, seizures, or paralysis. Although numerous medications have been used to treat MS, the medications have by and large been directed to mitigating the symptoms (e.g., pain, spasms, inflammation, etc.) as they occur, but no significant long-term recovery from MS has of yet been achieved, particularly for later MS. In view of the debilitating nature of MS, there would be a benefit in a permanent or long-term treatment that could more directly mitigate or even reverse the underlying cause of MS, which is demyelination of nerve tissue.

### SUMMARY

The present disclosure is directed to a method for treating or preventing multiple sclerosis (MS), which may be any of the types of MS known, including mild (or early stage) MS or advanced (or late stage) MS. The method of treatment may result in slowing, stabilizing, or even reversing demyelination (i.e., by remyelination), all of which can be considered mitigation of demyelination. In some embodiments, the mitigation of demyelination corresponds to a slowing of the progress of demyelination, the halting of demyelination, or even reversal of demyelination (i.e., remyelination) in nerve tissue of the subject that has undergone demyelination, for early stage, intermediate stage, or late stage MS. The remyelination may be achieved by the ability of a PKC activator (e.g., a bryostatin or bryolog) to mobilize a growth factor (e.g., BDNF, NGF, HGF, IGF, or the like) or activate oligodendrocytes that mobilize a growth factor to result in remyelination of axons. The PKC activator (e.g., a bryostatin or bryolog) may manipulate the activity of growth factor signaling pathways to improve the ability of endogenous oligodendrocyte progenitor (OP) cells to achieve remyelination. The PKC activator may regulate OP proliferation, differentiation, and survival in demyelinated lesions. In some embodiments, the PKC activator initiates, promotes, or activates oligodendrocyte-growth factor-mediated remyelination. The present disclosure is also directed to reduction of initial inflammation (particularly neuroinflammation) that occurs in MS, particularly early MS, by administration of a PKC activator, such as a bryostatin or bryolog. In some embodiments, the patient being treated with the PKC activator is not administered an NMDA receptor antagonist (i.e., the treatment excludes administration of a NMDA receptor antagonist).

In the method, a subject afflicted with mild or advanced MS or who exhibits possible early indicators of MS is administered a PKC activator (i.e. PKC activating compound, such as a bryostatin or bryolog compound) in a therapeutically effective amount to result in mitigation of demyelination or initiation of remyelination and the attenuation or prevention of one or more symptoms (e.g., inflammation) of the multiple sclerosis. In some embodiments, the method results in regeneration of lost synaptic connections in the brain of the subject (i.e., by inducing synaptogenesis), particularly in subjects with later (i.e., late stage or advanced) MS. As further discussed below, the PKC activating compound may be, for example, a macrocyclic lactone compound, such as a bryostatin compound (e.g., bryostatin-1) or bryolog compound, a polyunsaturated fatty acid (PUFA) or cyclopropanated or epoxidized derivative thereof, or a growth factor (e.g., BDNF, HGF, NGF, and IGF) or growth factor activating compound.

In some embodiments, the PKC activating compound is administered in an initial loading dose that is 15-25% greater in dosage than successive weekly dosages. In a first embodiment, the PKC activating compound is administered at an initial loading dose of about 15 micrograms per week for two consecutive weeks followed by about 12 micrograms on alternate weeks for a least four, six, eight, ten, or twelve weeks. In a second embodiment, the PKC activating compound is administered at an initial loading dose of about 24 micrograms per week for two consecutive weeks followed by about 20 micrograms on alternate weeks for at least four weeks. In a third embodiment, the PKC activating compound is administered at an initial loading dose of about 48 micrograms per week for two consecutive weeks followed by about 40 micrograms on alternate weeks for a least four, six, eight, ten, or twelve weeks.

### DETAILED DESCRIPTION

The present disclosure is foremost directed to a method for treating or preventing MS, more particularly by mitigating (e.g., stabilizing, halting, or even reversing) demyelination, or more particularly, by initiating, promoting, or activating remyelination. In some embodiments, the PKC activator initiates, promotes, or activates oligodendrocyte-growth factor-mediated remyelination. The PKC activator may more particularly manipulate the activity of growth factor signaling pathways to significantly improve the ability of endogenous oligodendrocyte progenitor (OP) cells to achieve extensive remyelination. In particular embodiments, the method results in remyelination of nerve cells of the subject that have undergone demyelination. In other embodiments, the method results in regeneration of lost synaptic connections in the brain of the subject. In particular embodiments, a PKC activator, such as a bryostatin or bryolog, is administered to a subject having later MS to mitigate or reverse synaptic loss in the brain of the subject. The present disclosure is also directed to reduction of initial inflammation (particularly neuroinflammation) that occurs in MS, particularly early MS, by administration of a PKC activator, such as a bryostatin (e.g., bryostatin-1) or bryolog, as further described below.

In the method, a subject is administered a pharmaceutically effective amount of a PKC activator to result in mitigation of demyelination, remyelination, or attenuation or prevention of one or more symptoms of the MS (e.g., neuroinflammation). The MS may be, more particularly, relapsing-remitting MS (RRMS), primary-progressive MS (PPMS), secondary-progressive MS (SPMS), and progressive-relapsing MS (PRMS). Any one of the foregoing disorders may also be deemed mild, moderate, or advanced.

NMDA receptor antagonists can have the negative effect of blocking the action of PKC activators, particularly bryostatins or bryologs. The principle targets of bryostatin, PKC isozymes, are known to regulate NMDA receptor functions (blocked by memantine, a well known NMDA receptor antagonist). Therefore, blockade of the NMDA receptor could offset most if not all of the bryostatin-induced SIB improvement. PKC regulation of the NMDA receptor functions includes increasing NMDA conductance by relieving Mg++ blockade, controlling trafficking of the NMDA receptor to the neuronal membranes, and enhancing NMDA-induced synaptogenesis. This synaptogenesis, a primary mechanism of action (MOA) of bryostatin demonstrated in a variety of pre-clinical models, is mediated by bryostatin-PKC epsilon enhancement of several synaptic growth factors that include BDNF, NGF, and IGF.

In view of the negative effects of NMDA receptor antagonists, in some embodiments, the patient being treated with the PKC activator is not administered an NMDA receptor antagonist (i.e., the treatment excludes administration of a NMDA receptor antagonist). In some embodiments, the PKC activator composition being administered to the patient is free of an NMDA receptor antagonist. As used herein, the term “NMDA receptor antagonist” refers to a substance that antagonizes (i.e., inhibits) the action of NMDA receptors. Some examples of NMDA receptor antagonists include memantine, drug combinations containing memantine, dextromethorphan, ketamine, phencyclidine (PCP), methoxetamine (MXE), and nitrous oxide (N₂O). Any one or all of the foregoing NMDA receptor antagonists may be excluded from the method of treating MS described herein.

As used herein, “protein kinase C activator” or “PKC activator” refers to a substance that increases the rate of the reaction catalyzed by PKC. PKC activators can be non-specific or specific activators. A specific activator activates one PKC isoform, e.g., PKC-ε (epsilon), to a greater detectable extent than another PKC isoform. Protein kinase C (PKC) is one of the largest gene families of protein kinase. Several PKC isozymes are expressed in the brain, including PKCα, PKCβ1, PKCβII, PKCδ, PKCε, and PKCγ. PKC is primarily a cytosolic protein, but with stimulation it translocates to the membrane.

PKC activators have been associated with various diseases and conditions. For example, PKC has been shown to be involved in numerous biochemical processes relevant to AD, and PKC activators have demonstrated neuroprotective activity in animal models of AD. PKC activation has a crucial role in learning and memory enhancement, and PKC activators have been shown to increase memory and learning (Sun and Alkon, Eur J Pharmacol. 2005; 512:43-51; Alkon et al., Proc Natl Acad Sci USA. 2005; 102:16432-16437). PKC activation also has been shown to induce synaptogenesis in rat hippocampus, suggesting the potential of PKC-mediated antiapoptosis and synaptogenesis during conditions of neurodegeneration (Sun and Alkon, Proc Natl Acad Sci USA. 2008; 105(36): 13620-13625). In fact, synaptic loss appears to be a pathological finding in the brain that is closely correlated with the degree of dementia in AD patients. PKC activation has further been shown to protect against traumatic brain injury-induced learning and memory deficits (Zohar et al., Neurobiology of Disease, 2011, 41: 329-337), has demonstrated neuroprotective activity in animal models of stroke (Sun et al., Eur. J. Pharmacol., 2005, 512: 43-51), and has shown neuroprotective activity in animal models of depression (Sun et al., Eur. J. Pharmacol., 2005, 512: 43-51).

Neurotrophins, particularly brain-derived neurotrophic factor (BDNF) and nerve growth factor (NGF), are key growth factors that initiate repair and regrowth of damaged neurons and synapses. Activation of some PKC isoforms, particularly PKCε and PKCα, protect against neurological injury, most likely by upregulating the production of neurotrophins such as BDNF (Weinreb et al., FASEB Journal. 2004; 18:1471-1473). The activation of PKCε also increases brain postsynaptic density anchoring protein (PSD-95) which is an important marker for synaptogenesis.

In addition, changes in dendritic spine density form the basis of learning- and memory-induced changes in synaptic structure that increase synaptic strength. Abnormalities in the number and morphology of dendritic spines have been observed in many cognitive disorders, such as attention deficit hyperactivity disorder, schizophrenia, autism, mental retardation, and fragile X syndrome. For example, the brains of schizophrenic patients and people suffering from cognitive-mood disorders show a reduced number of dendritic spines in the brain areas associated with these diseases. In mental retardation and autism, the shapes of the dendritic spines are longer and appear more immature.

As used herein, the term “fatty acid” refers to a compound composed of a hydrocarbon chain and ending in a free acid, an acid salt, or an ester. When not specified, the term “fatty acid” is meant to encompass all three forms. Those skilled in the art understand that certain expressions are interchangeable. For example, “methyl ester of linolenic acid” is the same as “linolenic acid methyl ester,” which is the same as “linolenic acid in the methyl ester form.”

As used herein, the term “cyclopropanated” or “CP” refers to a compound wherein at least one carbon-carbon double bond in the molecule has been replaced with a cyclopropane ring. The cyclopropyl group may be in cis or trans configuration. Unless otherwise indicated, the cyclopropyl group is in the cis configuration. Compounds with multiple carbon-carbon double bonds have many cyclopropanated forms. For example, a polyunsaturated compound in which only one double bond has been cyclopropanated is herein referred to as being in “CP1 form.” Similarly, “CP6 form” indicates that six double bonds are cyclopropanated. Docosahexaenoic acid (“DHA”) methyl ester has six carbon-carbon double bonds, and thus, can have 1-6 cyclopropane rings.

Shown below are the CP1 and CP6 forms. With respect to compounds that are not completely cyclopropanated (e.g. DHA-CP1), the cyclopropane group(s) can occur at any of the carbon-carbon double bonds.

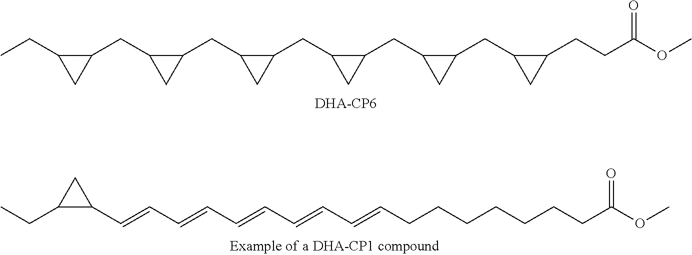

As used herein, the term “HGF activator” refers to a substance that increases the rate of the reaction catalyzed by HGF. HGF is well known in the art, as described in, for example, T. Nakamura et al., Proc., Jpn. Acad. Ser. B Phys. Biol. Sci., 86(6), 588-610, 2010. In some embodiments, the PKC activator is an HGF activator.

As used herein, the term “cholesterol” refers to cholesterol and derivatives thereof. For example, “cholesterol” may or may not include the dihydrocholesterol species.

As used herein, the word “synaptogenesis” refers to a process involving the formation of synapses. As used herein, the word “synaptic networks” refer to a multiplicity of neurons and synaptic connections between the individual neurons. Synaptic networks may include extensive branching with multiple interactions. Synaptic networks can be recognized, for example, by confocal visualization, electron microscopic visualization, and electrophysiologic recordings.

The phrase “pharmaceutically acceptable” refers to molecular entities and compositions that are physiologically tolerable and do not typically produce adverse reactions when administered to a subject. The pharmaceutically acceptable substance is typically approved by a regulatory agency or listed in the U.S. Pharmacopeia or other generally recognized pharmacopeia for use in animals, and more particularly in humans. The term “pharmaceutically acceptable carrier” generally refers to a liquid or solid chemical substance in which the active ingredient may be combined and which, following the combination, can be used to deliver the active ingredient to a subject. The carrier can also be, for example, a diluent, adjuvant, excipient, or vehicle for the compound being administered.

The term “therapeutically effective amount” refers to an amount of a therapeutic agent that results in a measurable or observable therapeutic response. A therapeutic response may be, for example, any response that a person of sound medical adjustment (e.g., a clinician or physician) will recognize as an effective response to the therapy, including improvement of symptoms and surrogate clinical markers. Thus, a therapeutic response will generally be a mitigation, amelioration, or inhibition of one or more symptoms of MS. A measurable therapeutic response may also include a finding that the disorder is prevented or has a delayed onset or is otherwise attenuated by the therapeutic agent.

The term “subject,” as used herein, refers to a human or other mammal having MS or at risk of developing MS or having one or more symptoms or conditions believed to be precursors of early MS. The one or more precursor symptoms may include vision problems associated with inflammation of the optic nerve (i.e., optic neuritis), vision problems (e.g., nystagmus or double vision), fatigue, numbness, tingling, cognitive decline, muscle spasms, sensory impairment, dysphagia, and incontinence. The early or precursor symptoms may also be initially diagnosed as clinically isolated syndrome (CIS), which may be monofocal episode or multifocal episode types of CIS. In some subjects, a diagnosis of early MS is confirmed by evidence indicating loss of myelin in nerve cells, such as may be determined by an MRI. Some examples of mammals other than humans that may be treated include dogs, cats, monkeys, and apes. In some embodiments, the subject has an age of 20-45 years. In other embodiments, the subject has an age of at least or over 45, 50, 55, or 60 years old.

The terms “approximately” and “about” mean to be nearly the same as a referenced number or value including an acceptable degree of error for the quantity measured given the nature or precision of the measurements. As used herein, the terms “approximately” and “about” are generally understood to encompass ±20% or ±10% of a specified amount, frequency or value. Numerical quantities given herein are approximate unless stated otherwise, meaning that the term “about” or “approximately” can be inferred when not expressly stated. For example, the term “about 20 μg” may be interpreted as precisely that amount or as being within a margin of 16-24 μg or 18-22 μg.

The terms “administer,” “administration,” or “administering,” as used herein, refer to (1) providing, giving, dosing and/or prescribing by either a health practitioner or his/her authorized agent or under his/her direction a composition according to the disclosure, and (2) putting into, taking, or consuming by the patient or person himself or herself, a composition according to the disclosure. As used herein, “administration” of a composition includes any route of administration, including oral, intravenous, subcutaneous, intraperitoneal, and intramuscular.

The phrase “weekly dosing regimen” is used when the subject is administered a dose of a therapeutic agent (drug) every week for a predetermined number of consecutive weeks. For example, the subject may receive a single dose of a therapeutic agent each week for three consecutive weeks.

The phrases “spaced dosing regimen” and “intermittent dosing regimen” are herein used interchangeably and refer to an “on/off” dosing regimen of a defined periodicity. In some embodiments, a spaced dosing regimen or intermittent dosing regimen may be used for administering a PKC activating compound to a subject. In some embodiments, intermittent dosing of a PKC activator results in restoring or upregulating BDNF, increasing the postsynaptic density of the anchoring protein PSD-95, and/or lowering or preventing the downregulation of PCK-ε.

The spaced or intermittent dosing regimen may entail, for example, administering a PKC activating compound to the subject once a week for two or three consecutive weeks, followed by cessation of administration or dosing for two or three consecutive weeks. In further embodiments, the administration may continue in alternating intervals of administering the PKC activator once a week for two or three consecutive weeks, followed by cessation of administration or dosing for two or three consecutive weeks, and continuing those alternating intervals over a period of about 4 months, about 8 months, about 1 year, about 2 years, about 5 years, or otherwise for the duration of therapy with the PKC activator.

The PKC activator may be administered according to any suitable dosing schedule or regimen. In some embodiments, the PKC activator, such as a bryostatin (e.g., bryostatin-1), may be administered in an amount ranging from about 0.01 μg/m² to about 100 μg/m². In different embodiments, the amount administered is precisely, about, up to, or less than 0.01 μg/m², 0.05 μg/m², 0.1 μg/m², 0.5 μg/m², 1 μg/m², 5 μg/m², 10 μg/m², 15 μg/m², 20 μg/m², 25 μg/m², 30 μg/m², 35 μg/m², 40 μg/m², 45 μg/m², 50 μg/m², 55 μg/m², 60 μg/m², 65 μg/m², 70 μg/m², 75 μg/m², 80 μg/m², 85 μg/m², 90 μg/m², 95 μg/m², or 100 μg/m², or an amount within a range bounded by any two of the foregoing amounts, e.g., 0.01-100 μg/m², 0.1-100 μg/m², 1-100 μg/m², 5-100 μg/m², 10-100 μg/m², 20-100 μg/m², 0.01-50 μg/m², 0.1-50 μg/m², 1-50 μg/m², 5-50 μg/m², 10-50 μg/m², 20-50 μg/m², 0.01-40 μg/m², 0.1-40 μg/m², 1-40 μg/m², 5-40 μg/m², 10-40 μg/m², 20-40 μg/m², 0.01-30 μg/m², 0.1-30 μg/m², 1-30 μg/m², 5-30 μg/m², 10-30 μg/m², 20-30 μg/m², 0.01-20 μg/m², 0.1-20 μg/m², 1-20 μg/m², 5-20 μg/m², or 10-20 μg/m². In particular embodiments, the amount may range from about 10-40 μg/m², or more particularly, about 15 μg/m², about 20 μg/m², about 25 μg/m², about 30 μg/m², about 35 μg/m², or about 40 μg/m², or about 45 μg/m², or about 50 μg/m², or an amount within a range bounded by any two of the foregoing values. Notably, any of the amounts above or below expressed as “μg/m²” may alternatively be interpreted in terms of micrograms (g) or micrograms per 50 kg body weight “μg/50 kg”. For example, 25 μg/m² may be interpreted as 25 μg or 25 μg/50 kg. In particular embodiments, any of the foregoing amounts (which may be within any of the exemplary ranges disclosed above) may be administered per day, every other day, once every three days, precisely or at least once per week, precisely or at least once every two weeks, and any of the foregoing administrations may be continued as a dosing regimen for at least one, two, three, four, five, six, seven, eight, nine, ten, eleven, or twelve weeks, or for at least four, five, six, seven, eight, nine, ten, eleven, or twelve months.

In some embodiments, the PKC activator is administered as a dose in the range of about 0.01 to 100 μg/m²/week. For example, the dose may be administered each week in a range of about 0.01 to about 25 μg/m²/week; about 1 to about 20 μg/m²/week, about 5 to about 20 μg/m²/week, or about 10 to about 20 μg/m²/week. In particular embodiments, the dose may be about or less than, for example, 5 μg/m²/week, 10 μg/m²/week, 15 μg/m²/week, 20 μg/m²/week, 25 μg/m²/week, or 20 μg/m²/week. Any of the foregoing dosages may be administered over a suitable time period, e.g., three weeks, four weeks, (approximately 1 month), two months, three months (approximately 12 or 13 weeks), four months, five months, six months, or a year. Notably, any of the amounts above or below expressed as “μg/m²” may alternatively correspond to micrograms (g) or micrograms per 50 kg body weight “μg/50 kg” per dosage or amount per week.

In some embodiments, the PKC activator (e.g., a bryostatin) is administered in an amount of precisely or about 20 μg, 30 μg, or 40 μg (20 μg/m², 30 μg/m², or 40 μg/m²) every week or every two weeks for a total period of time of, e.g., four weeks, (approximately 1 month), five weeks, six weeks, eight weeks, ten weeks, twelve weeks, four months, five months, six months, or a year. The administration may alternatively start with an initial single higher amount (e.g., 10%, 15%, 20%, or 25% higher amount than successive administrations). For example, in some embodiments, the PKC activator may be administered in an amount of precisely or about 15 μg, 24 μg, or 48 μg for the first week or first two or three consecutive weeks followed by administrations of 12 μg, 20 μg or 40 μg, respectively, every week or alternately every two or three weeks for at least four weeks (approximately 1 month), five weeks, six weeks, eight weeks, ten weeks, twelve weeks, fifteen weeks, eighteen weeks, or for at least three months, four months, five months, six months, or a year. The term “alternately,” as used herein, indicates a period of time in which the PKC activator is not being administered. For example, “alternately every two or three weeks” indicates, respectively, regular one-week periods of no administration or regular two-week periods of no administration (also referred to herein as “1 on/1 off” and “1 on/2 off” dosing regimens). Other alternating dosing regimens are possible, including, for example, “2 on/1 off”, “2 on/2 off”, “1 on/3 off”, “2 on/3 off”, “3 on/3 off”, “3 on/1 off”, and “3 on/2 off”. Notably, any of the amounts above or below expressed as μg may alternatively be interpreted in terms of μg/m² or micrograms per 50 kg body weight “μg/50 kg”.

In some embodiments, before administering the PKC activator, the subject is first confirmed to have MS or an associated disorder or an early sign or precursor of MS. The subject may be confirmed to have MS by, for example, undergoing a blood test for one or more biomarkers associated with MS, an MRI, an evoked potential test, or a spinal tap fluid test. The subject may be considered to be at risk or likely to develop MS if the subject is confirmed to have optic neuritis, clinically isolated syndrome, detection of brain lesions by MRI, and/or presence of oligoclonal bands in the subject's cerebrospinal fluid. In some cases, confirmation of optic neuritis or other neurological inflammation may be considered a positive indicator or positive risk factor for MS.

In some embodiments, the PKC activator is selected from macrocyclic lactones, bryostatins, bryologs, diacylglycerols, isoprenoids, octylindolactam, gnidimacrin, ingenol, iripallidal, napthalenesulfonamides, diacylglycerol inhibitors, growth factors, polyunsaturated fatty acids, monounsaturated fatty acids, cyclopropanated polyunsaturated fatty acids, cyclopropanated monounsaturated fatty acids, fatty acids alcohols and derivatives, and fatty acid esters.

In some embodiments, the therapeutically effective amount of PKC activator is administered according to any suitable dosing schedule or regimen described. For purposes of the present invention, the administration of the PKC activator should result in at least a stabilization or halting of demyelination or inflammation of neural tissue, and more preferably, a reduction or reversal of demyelination or inflammation of neural tissue. The administration may alternatively prevent such demyelination or inflammation from occurring or from worsening. In some embodiments, the therapeutically effective amount of PKC activator is administered in a suitable dosing schedule to result in remyelination. The remyelination may be achieved by the ability of a PKC activator (e.g., a bryostatin or bryolog) to mobilize a growth factor (e.g., BDNF, NGF, HGF, IGF, or the like) or activate oligodendrocytes that mobilize a growth factor to result in remyelination of axons. In some embodiments, the PKC activator stimulates proliferation of immature OP cells followed by differentiation into myelinating oligodendrocytes to result in extensive remyelination. The PKC activator may stimulate OP cells to generate oligodendrocytes. The PKC activator may stimulate OP proliferation to promote more rapid remyelination with resultant enhancement in remyelination in areas with low OP density. The therapeutically effective amount of PKC activator may also result in reduction of initial inflammation (particularly neuroinflammation) that occurs in MS, particularly early MS.

In some embodiments, administration of the PKC activator also results in an increase in the number of fully mature mushroom spine synapses. In other embodiments, administration of the PKC activator results in at least partial or full restoration of mature mushroom spines or mushroom spine synapses. In other embodiments, administration of the PKC activator results in regeneration of lost synaptic connections in the brain of the subject.

In different embodiments, the PKC activator is selected from macrocyclic lactones, bryostatins, bryologs, diacylglycerols, isoprenoids, octylindolactam, gnidimacrin, ingenol, iripallidal, napthalenesulfonamides, diacylglycerol inhibitors, growth factors, growth factor activators, polyunsaturated fatty acids, monounsaturated fatty acids, cyclopropanated polyunsaturated fatty acids, cyclopropanated monounsaturated fatty acids, fatty acid alcohols and derivatives, and fatty acid esters. In particular embodiments, the PKC activator is a macrocyclic lactone selected from bryostatins and neristatin, such as neristatin-1. In a further embodiment, the PKC activator is a bryostatin, such as bryostatin-1, bryostatin-2, bryostatin-3, bryostatin-4, bryostatin-5, bryostatin-6, bryostatin-7, bryostatin-8, bryostatin-9, bryostatin-10, bryostatin-11, bryostatin-12, bryostatin-13, bryostatin-14, bryostatin-15, bryostatin-16, bryostatin-17, or bryostatin-18. In a further embodiment, the PKC activator is bryostatin-1. In one embodiment, the therapeutically effective amount of the PKC activator, such as bryostatin-1, is about 25 μg/m².

In some embodiments, the PKC activator is a macrocyclic lactone. Macrocyclic lactones (also known as macrolides) generally comprise 14-, 15-, or 16-membered lactone rings. Macrolides belong to the polyketide class of natural products. Macrocyclic lactones and derivatives thereof are described, for example, in U.S. Pat. Nos. 6,187,568; 6,043,270; 5,393,897; 5,072,004; 5,196,447; 4,833,257; and 4,611,066; and 4,560,774; each incorporated by reference herein in its entirety. Those patents describe various compounds and various uses for macrocyclic lactones including their use as an anti-inflammatory or anti-tumor agents. See also Szallasi et al. J. Biol. Chem. (1994), vol. 269, pp. 2118-2124; Zhang et al., Cancer Res. (1996), vol. 56, pp. 802-808; Hennings et al. Carcinogenesis (1987), vol. 8, pp. 1343-1346; Varterasian et al. Clin. Cancer Res. (2000), vol. 6, pp. 825-828; Mutter et al. Bioorganic & Med. Chem. (2000), vol. 8, pp. 1841-1860; each incorporated by reference herein in its entirety. In particular embodiments, the macrocyclic lactone is a bryostatin. Bryostatins include, for example, Bryostatin-1, Bryostatin-2, Bryostatin-3, Bryostatin-4, Bryostatin-5, Bryostatin-6, Bryostatin-7, Bryostatin-8, Bryostatin-9, Bryostatin-10, Bryostatin-11, Bryostatin-12, Bryostatin-13, Bryostatin-14, Bryostatin-15, Bryostatin-16, Bryostatin-17, and Bryostatin-18. In particular embodiments, the PKC activator is a macrocyclic lactone selected from bryostatin and neristatin, such as neristatin-1.

In one embodiment, the bryostatin is or includes Bryostatin-1 (shown below).

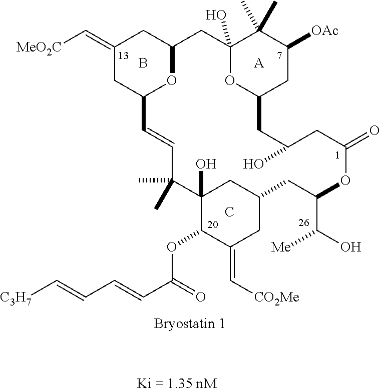

In another embodiment, the bryostatin is or includes Bryostatin-2 (shown below; R═COC₇H₁₁, R′═H).

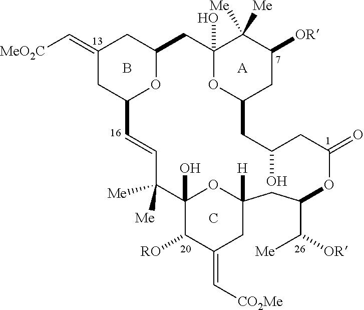

In another embodiment, the macrocyclic lactone is or includes a neristatin, such as neristatin-1. In another embodiment, the macrocyclic lactone is selected from macrocyclic derivatives of cyclopropanated PUFAs such as, 24-octaheptacyclononacosan-25-one (cyclic DHA-CP6) (shown below).

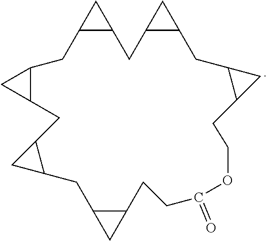 [SMILES: O=C1CCC2CC2CC2CC2CC2CC2CC2CC2CC2CC2CC2CC2CCO1]

In another embodiment, the macrocyclic lactone is or includes a bryolog, wherein bryologs are analogues of bryostatin. Bryologs can be chemically synthesized or produced by certain bacteria. Different bryologs exist that modify, for example, the rings A, B, and C (see Bryostatin-1, figure shown above) as well as the various substituents. Generally, bryologs are considered less specific and less potent than bryostatin but are easier to prepare.

Table 1 summarizes structural characteristics of several bryologs and their affinity for PKC (ranging from 0.25 nM to 10 μM). While Bryostatin-1 has two pyran rings and one 6-membered cyclic acetal, in most bryologs one of the pyrans of Bryostatin-1 is replaced with a second 6-membered acetal ring. This modification may reduce the stability of bryologs, relative to Bryostatin-1, for example, in strong acid or base, but has little significance at physiological pH. Bryologs also tend to have a lower molecular weight (ranging from about 600 g/mol to 755 g/mol), as compared to Bryostatin-1 (988), a property which may facilitate transport across the blood-brain barrier.

TABLE 1
Bryologs

| | PKC Affin | | |
|---|---|---|---|
| Name | (nM) | MW | Description |
| Bryostatin-1 | 1.35 | 988 | 2 pyran + 1 cyclic acetal + |
| | | | macrocycle |
| Analog 1 | 0.25 | 737 | 1 pyran + 2 cyclic acetal + |
| | | | macrocycle |
| Analog 2 | 6.50 | 723 | 1 pyran + 2 cyclic acetal + |
| | | | macrocycle |
| Analog 7a | — | 642 | 1 pyran + 2 cyclic acetals + |
| | | | macrocycle |
| Analog 7b | 297 | 711 | 1 pyran + 2 cyclic acetals + |
| | | | macrocycle |
| Analog 7c | 3.4 | 726 | 1 pyran + 2 cyclic acetals + |
| | | | macrocycle |
| Analog 7d | 10000 | 745 | 1 pyran + 2 cyclic acetals + |
| | | | macrocycle, acetylated |
| Analog 8 | 8.3 | 754 | 2 cyclic acetals + macrocycle |
| Analog 9 | 10000 | 599 | 2 cyclic acetals |

Analog 1 exhibits the highest affinity for PKC. Wender et al., Curr. Drug Discov. Technol. (2004), vol. 1, pp. 1-11; Wender et al. Proc. Natl. Acad. Sci. (1998), vol. 95, pp. 6624-6629; Wender et al., J. Am. Chem. Soc. (2002), vol. 124, pp. 13648-13649, each incorporated by reference herein in their entireties. Only Analog 1 exhibits a higher affinity for PKC than Bryostatin-1. Analog 2, which lacks the A ring of Bryostatin-1, is the simplest analog that maintains high affinity for PKC. In addition to the active bryologs, Analog 7d, which is acetylated at position 26, has virtually no affinity for PKC.

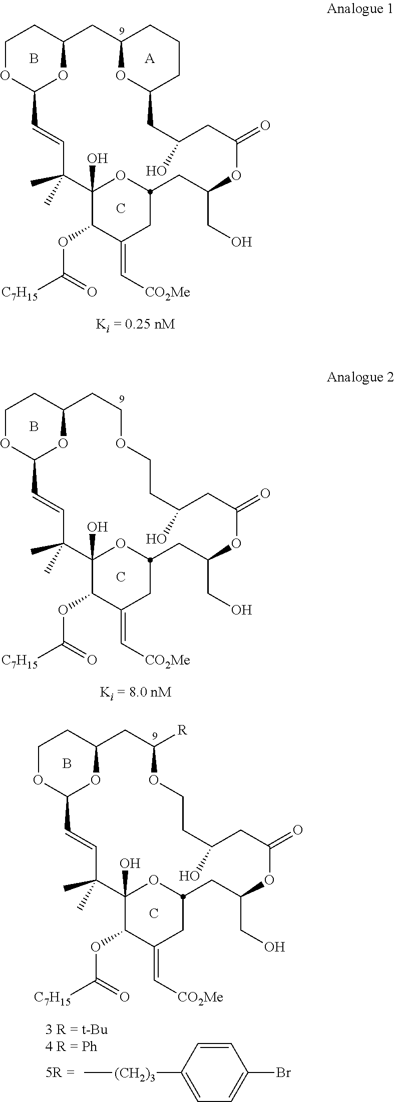

B-ring bryologs may also be used in the present disclosure. These synthetic bryologs have affinities in the low nanomolar range. Wender et al., Org Lett. (2006), vol. 8, pp. 5299-5302, incorporated by reference herein in its entirety. B-ring bryologs have the advantage of being completely synthetic, and do not require purification from a natural source.

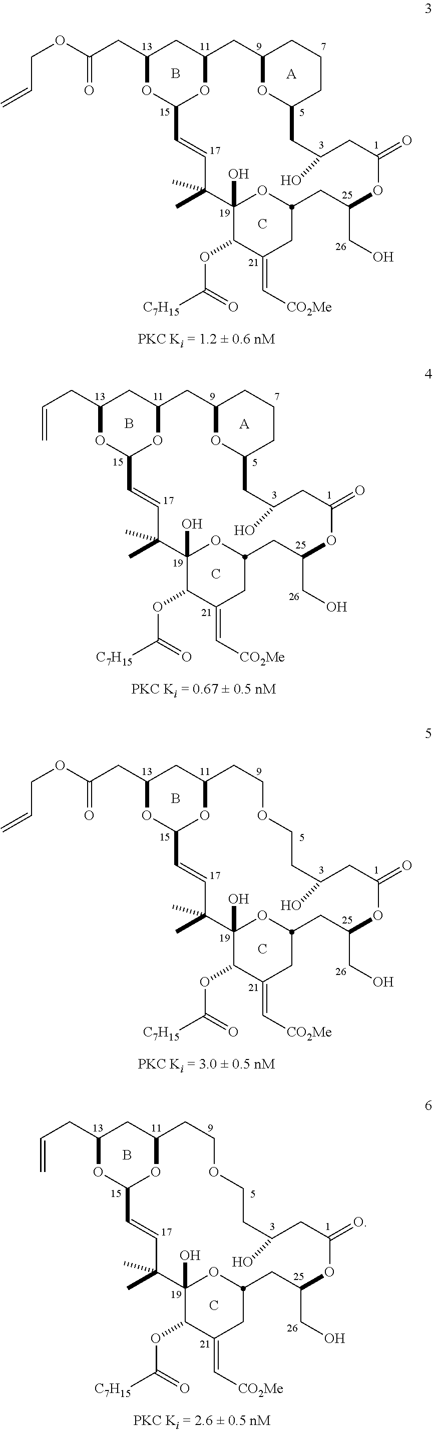

A third class of suitable bryostatin analogs are the A-ring bryologs. These bryologs generally have slightly lower affinity for PKC than Bryostatin-1 (6.5 nM, 2.3 nM, and 1.9 nM for bryologs 3, 4, and 5, respectively) and a lower molecular weight. A-ring substituents are important for non-tumorigenesis.

Bryostatin analogs are described, for example, in U.S. Pat. Nos. 6,624,189 and 7,256,286. Methods using macrocyclic lactones to improve cognitive ability are also described in U.S. Pat. No. 6,825,229 B2.

In some embodiments, a bryostatin (e.g., bryostatin-1) or bryolog is administered to a subject having early MS or at risk for MS or having precursor symptoms indicative of the onset of early MS, and the bryostatin or bryolog is efficacious in reducing initial inflammation (particularly neuroinflammation) that occurs in MS, particularly early MS. The bryostatin or bryolog may also be efficacious in remyelinating axons that have undergone some level of demyelination. In some embodiments, the bryostatin or bryolog is administered in the absence of an NMDA receptor antagonist.

The PKC activator may also include derivatives of diacylglycerols (DAGs). See, e.g., Niedel et al., Proc. Natl. Acad. Sci. (1983), vol. 80, pp. 36-40; Mori et al., J. Biochem. (1982), vol. 91, pp. 427-431; Kaibuchi et al., J. Biol. Chem. (1983), vol. 258, pp. 6701-6704. Activation of PKC by diacylglycerols is transient, because they are rapidly metabolized by diacylglycerol kinase and lipase. Bishop et al. J. Biol. Chem. (1986), vol. 261, pp. 6993-7000; Chuang et al. Am. J. Physiol. (1993), vol. 265, pp. C927-C933; incorporated by reference herein in their entireties. The fatty acid substitution on the diacylglycerol derivatives may determine the strength of activation. Diacylglycerols having an unsaturated fatty acid may be most active. The stereoisomeric configuration is important; fatty acids with a 1,2-sn configuration may be active while 2,3-sn-diacylglycerols and 1,3-diacylglycerols may not bind to PKC. Cis-unsaturated fatty acids may be synergistic with diacylglycerols. In some embodiments, the PKC activator excludes DAG or DAG derivatives.

The PKC activator may also include isoprenoids. Farnesyl thiotriazole, for example, is a synthetic isoprenoid that activates PKC with a K_{d} of 2.5 μM. Farnesyl thiotriazole, for example, is equipotent with dioleoylglycerol, but does not possess hydrolyzable esters of fatty acids. Gilbert et al., Biochemistry (1995), vol. 34, pp. 3916-3920; incorporated by reference herein in its entirety. Farnesyl thiotriazole and related compounds represent a stable, persistent PKC activator. Because of its low molecular weight (305.5 g/mol) and absence of charged groups, farnesyl thiotriazole may readily cross the blood-brain barrier.

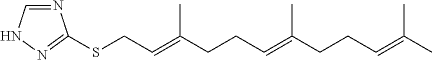 [SMILES: CC(C)=CCC/C(C)=C/CC/C(C)=C/CSc1nc[nH]n1]

Yet other types of PKC activators include octylindolactam V, gnidimacrin, and ingenol. Octylindolactam V is a non-phorbol protein kinase C activator related to teleocidin. The advantages of octylindolactam V (specifically the (−)-enantiomer) may include greater metabolic stability, high potency (EC₅₀=29 nM) and low molecular weight that facilitates transport across the blood brain barrier. Fujiki et al. Adv. Cancer Res. (1987), vol. 49 pp. 223-264; Collins et al. Biochem. Biophys. Res. Commun. (1982), vol. 104, pp. 1159-4166, each incorporated by reference herein in its entirety.

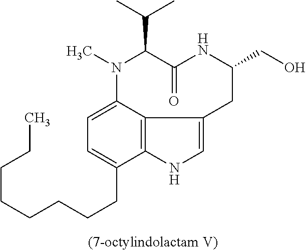 [SMILES: CCCCCCCCc1ccc2c3c(c[nH]c13)C[C@@H](CO)NC(=O)[C@H](C(C)C)N2C]

Gnidimacrin is a daphnane-type diterpene that displays potent antitumor activity at concentrations of 0.1 nM-1 nM against murine leukemias and solid tumors. It may act as a PKC activator at a concentration of 0.3 nM in K562 cells, and regulate cell cycle progression at the G1/S phase through the suppression of Cdc25A and subsequent inhibition of cyclin-dependent kinase 2 (Cdk2) (100% inhibition achieved at 5 ng/ml). Gnidimacrin is a heterocyclic natural product similar to Bryostatin-1, but somewhat smaller (MW=774.9 g/mol).

Iripallidal is a bicyclic triterpenoid isolated from Iris pallida. Iripallidal displays anti-proliferative activity in a NCI 60 cell line screen with GI₅₀ (concentration required to inhibit growth by 50%) values from micromolar to nanomolar range. It binds to PKCa with high affinity (Kᵢ=75.6 nM). It may induce phosphorylation of Erkl/2 in a RasGRP3-dependent manner. Its molecular weight is 486.7 g/mol. Iripallidal is about half the size of Biyostatin-1 and lacks charged groups.

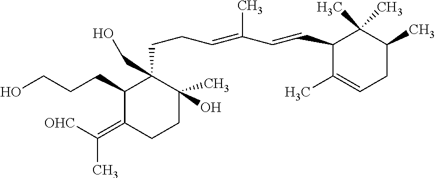

Ingenol is a diterpenoid related to phorbol but less toxic. It is derived from the milkweed plant Euphorbia peplus. Ingenol 3,20-dibenzoate, for example, competes with [3H] phorbol dibutyrate for binding to PKC (Kᵢ=240 nM). Winkler et al., J. Org. Chem. (1995), vol. 60, pp. 1381-1390, incorporated by reference herein. Ingenol-3-angelate exhibits antitumor activity against squamous cell carcinoma and melanoma when used topically. Ogbourne et al. Anticancer Drugs (2007), vol. 18, pp. 357-362, incorporated by reference herein.

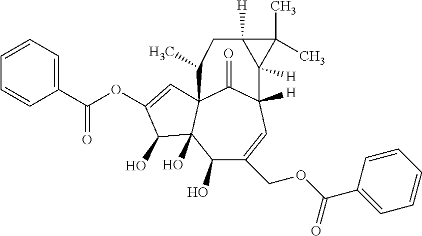 [SMILES: C[C@@H]1C[C@@H]2[C@H]([C@@H]3C=C(COC(=O)c4ccccc4)[C@@H](O)[C@]4(O)[C@@H](O)C(OC(=O)c5ccccc5)=C[C@@]14C3=O)C2(C)C]

The PKC activator may also include the class of napthalenesulfonamides, including N-(n-heptyl)-5-chloro-1-naphthalenesulfonamide (SC-10) and N-(6-phenylhexyl)-5-chloro-1-naphthalenesulfonamide. SC-10 may activate PKC in a calcium-dependent manner, using a mechanism similar to that of phosphatidylserine. Ito et al., Biochemistry (1986), vol. 25, pp. 4179-4184, incorporated by reference herein. Naphthalenesulfonamides act by a different mechanism than bryostatin and may show a synergistic effect with bryostatin or member of another class of PKC activators. Structurally, naphthalenesulfonamides are similar to the calmodulin (CaM) antagonist W-7, but are reported to have no effect on CaM kinase.

The PKC activator may also include the class of diacylglycerol kinase inhibitors, which indirectly activate PKC. Examples of diacylglycerol kinase inhibitors include, but are not limited to, 6-(2-(4-[(4-fluorophenyl)phenylmethylene]-1-piperidinyl)ethyl)-7-methyl-5H-thiazolo[3,2-a]pyrimidin-5-one (R59022) and [3-[2-[4-(bis-(4-fluorophenyl)methylene]piperidin-1-yl)ethyl]-2,3-dihydro-2-thioxo-4(1H)-quinazolinone (R59949).

The PKC activator may also be a growth factor, such as fibroblast growth factor 18 (FGF-18) and insulin growth factor, which function through the PKC pathway. FGF-18 expression is up-regulated in learning, and receptors for insulin growth factor have been implicated in learning. Activation of the PKC signaling pathway by these or other growth factors offers an additional potential means of activating PKC.

The PKC activator may or may not also include a hormone or growth factor activator, e.g., a 4-methyl catechol derivative, such as 4-methylcatechol acetic acid (MCBA), which stimulates the synthesis and/or activation of growth factors, such as NGF and BDNF. In turn, NGF and BDNF activate PKC as well as convergent pathways responsible for synaptogenesis and/or neuritic branching.

The PKC activator may also be a polyunsaturated fatty acid (“PUFA”). These compounds are essential components of the nervous system and have numerous health benefits. In general, PUFAs increase membrane fluidity, rapidly oxidize to highly bioactive products, produce a variety of inflammatory and hormonal effects, and are rapidly degraded and metabolized. The inflammatory effects and rapid metabolism is likely the result of their active carbon-carbon double bonds.

In one embodiment, the PUFA is selected from linoleic acid (shown below).

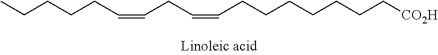 [SMILES: CCCCC/C=C\C/C=C\CCCCCCCC(=O)O]

The PKC activator may also be a PUFA or MUFA derivative. In particular embodiments, the PUFA or MUFA derivative is a cyclopropanated derivative. Certain cyclopropanated PUFAs, such as DCPLA (i.e., linoleic acid with cyclopropane at both double bonds), may be able to selectively activate PKC-ε. See Journal of Biological Chemistry, 2009, 284(50): 34514-34521; see also U.S. Patent Application Publication No. 2010/0022645 A1. Like their parent molecules, PUFA derivatives are thought to activate PKC by binding to the PS site.

Cyclopropanated fatty acids exhibit low toxicity and are readily imported into the brain where they exhibit a long half-life (t_{1/2}). Conversion of the double bonds into cyclopropane rings prevents oxidation and metabolism to inflammatory byproducts and creates a more rigid U-shaped 3D structure that may result in greater PKC activation. Moreover, this U-shape may result in greater isoform specificity. For example, cyclopropanated fatty acids may exhibit potent and selective activation of PKC-ε.

The Simmons-Smith cyclopropanation reaction is an efficient way of converting double bonds to cyclopropane groups. This reaction, acting through a carbenoid intermediate, preserves the cis-stereochemistry of the parent molecule. Thus, the PKC-activating properties are increased while metabolism into other molecules, such as bioreactive eicosanoids, thromboxanes, or prostaglandins, is prevented.

A particular class of PKC-activating fatty acids is omega-3 PUFA derivatives. In at least one embodiment, the omega-3 PUFA derivatives are selected from cyclopropanated docosahexaenoic acid, cyclopropanated eicosapentaenoic acid, cyclopropanated rumelenic acid, cyclopropanated parinaric acid, and cyclopropanated linolenic acid (CP3 form shown below).

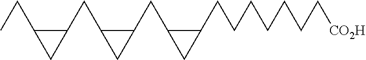 [SMILES: CCC1CC1CC1CC1CC1CC1CCCCCCCC(=O)O]

Another class of PKC-activating fatty acids is omega-6 PUFA derivatives. In at least one embodiment, the omega-6 PUFA derivatives are selected from cyclopropanated linoleic acid (“DCPLA,” CP2 form shown below),

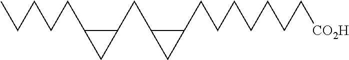 [SMILES: CCCCCC1CC1CC1CC1CCCCCCCC(=O)O]

cyclopropanated arachidonic acid, cyclopropanated eicosadienoic acid, cyclopropanated dihomo-gamma-linolenic acid, cyclopropanated docosadienoic acid, cyclopropanated adrenic acid, cyclopropanated calendic acid, cyclopropanated docosapentaenoic acid, cyclopropanated jacaric acid, cyclopropanated pinolenic acid, cyclopropanated podocarpic acid, cyclopropanated tetracosatetraenoic acid, and cyclopropanated tetracosapentaenoic acid.

Vernolic acid is a naturally occurring compound. However, it is an epoxyl derivative of linoleic acid and therefore, as used herein, is considered an omega-6 PUFA derivative. In addition to vernolic acid, cyclopropanated vernolic acid (shown below) is an omega-6 PUFA derivative.

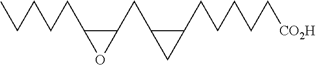 [SMILES: CCCCCC1OC1CC1CC1CCCCCC(=O)O]

Another class of PKC-activating fatty acids is the class of omega-9 PUFA derivatives. In at least one embodiment, the omega-9 PUFA derivatives are selected from cyclopropanated eicosenoic acid, cyclopropanated mead acid, cyclopropanated erucic acid, and cyclopropanated nervonic acid.

Yet another class of PKC-activating fatty acids is the class of monounsaturated fatty acid (“MUFA”) derivatives. In at least one embodiment, the MUFA derivatives are selected from cyclopropanated oleic acid (shown below),

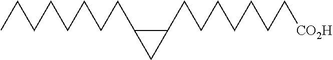 [SMILES: CCCCCCCCC1CC1CCCCCCCC(=O)O]

and cyclopropanated elaidic acid (shown below).

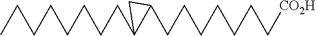 [SMILES: CCCCCCCCC1CC1CCCCCCCC(=O)O]

PKC-activating MUFA derivatives include epoxylated compounds such as trans-9,10-epoxystearic acid (shown below).

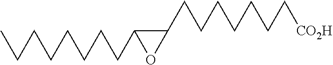 [SMILES: CCCCCCCCC1OC1CCCCCCCC(=O)O]

Another class of PKC-activating fatty acids is the class of omega-5 and omega-7 PUFA derivatives. In at least one embodiment, the omega-5 and omega-7 PUFA derivatives are selected from cyclopropanated rumenic acid, cyclopropanated alpha-elostearic acid, cyclopropanated catalpic acid, and cyclopropanated punicic acid.

Another class of PKC activators is the class of fatty acid alcohols and derivatives thereof, such as cyclopropanated PUFA and MUFA fatty alcohols. It is thought that these alcohols activate PKC by binding to the PS site. These alcohols can be derived from different classes of fatty acids.

In at least one embodiment, the PKC-activating fatty alcohols are derived from omega-3 PUFAs, omega-6 PUFAs, omega-9 PUFAs, and MUFAs, especially the fatty acids noted above. In at least one embodiment, the fatty alcohol is selected from cyclopropanated linolenyl alcohol (CP3 form shown above), cyclopropanated linoleyl alcohol (CP2 form shown above), cyclopropanated elaidic alcohol (shown above), cyclopropanated DCPLA alcohol, and cyclopropanated oleyl alcohol.

Another class of PKC activators includes fatty acid esters and derivatives thereof, such as cyclopropanated PUFA and MUFA fatty esters. In at least one embodiment, the cyclopropanated fatty esters are derived from omega-3 PUFAs, omega-6 PUFAs, omega-9 PUFAs, MUFAs, omega-5 PUFAs, and omega-7 PUFAs. These compounds are thought to activate PKC through binding on the PS site. One advantage of such esters is that they are generally considered to be more stable that their free acid counterparts.

In one embodiment, the PKC-activating fatty acid esters derived from omega-3 PUFAs are selected from cyclopropanated eicosapentaenoic acid methyl ester (CP5 form shown below)

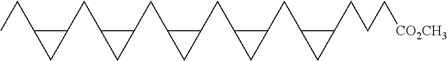 [SMILES: CCC1CC1CC1CC1CC1CC1CC1CC1CC1CC1CCCC(=O)OC]

and cyclopropanated linolenic acid methyl ester (CP3 form shown below).

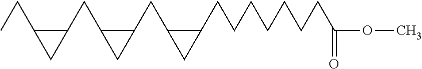 [SMILES: CCC1CC1CC1CC1CC1CC1CCCCCCCC(=O)OC]

In another embodiment, the omega-3 PUFA esters are selected from esters of DHA-CP6 and aliphatic and aromatic alcohols. In at least one embodiment, the ester is cyclopropanated docosahexaenoic acid methyl ester (CP6 form shown below).

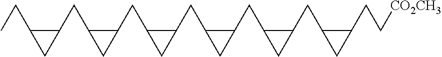 [SMILES: CCC1CC1CC1CC1CC1CC1CC1CC1CC1CC1CC1CC1CCC(=O)OC]

In one embodiment, PKC-activating fatty esters derived from omega-6 PUFAs are selected from cyclopropanated arachidonic acid methyl ester (CP4 form shown below),

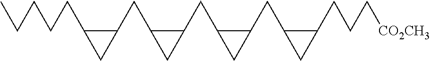 [SMILES: CCCCCC1CC1CC1CC1CC1CC1CC1CC1CCCC(=O)OC]

cyclopropanated vernolic acid methyl ester (CP1 form shown below), and

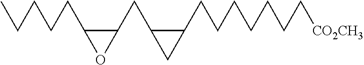 [SMILES: CCCCCC1OC1CC1CC1CCCCCCCC(=O)OC]

vernolic acid methyl ester (shown below).

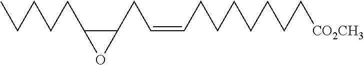 [SMILES: CCCCCC1OC1C/C=C\CCCCCCCC(=O)OC]

In particular embodiments, the PKC activating compound is an ester derivative of DCPLA (CP6-linoleic acid). In one embodiment, the ester of DCPLA is an alkyl ester. The alkyl group of the DCPLA alkyl esters may be linear, branched, and/or cyclic. The alkyl groups may be saturated or unsaturated. When the alkyl group is an unsaturated cyclic alkyl group, the cyclic alkyl group may be aromatic. The alkyl group may be selected from, for example, methyl, ethyl, propyl (e.g., isopropyl), and butyl (e.g., tert-butyl) esters. DCPLA in the methyl ester form (“DCPLA-ME”) is shown below.

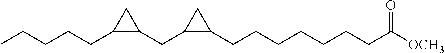 [SMILES: CCCCCC1CC1CC1CC1CCCCCCCC(=O)OC]

In another embodiment, the esters of DCPLA are derived from a benzyl alcohol (unsubstituted benzyl alcohol ester shown below). In yet another embodiment, the esters of DCPLA are derived from aromatic alcohols such as phenols used as antioxidants and natural phenols with pro-learning ability. Some specific examples include estradiol, butylated hydroxytoluene, resveratrol, polyhydroxylated aromatic compounds, and curcumin.

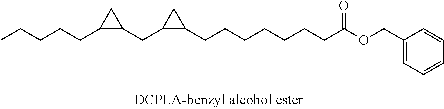 [SMILES: CCCCCC1CC1CC1CC1CCCCCCCC(=O)OCc1ccccc1]

Another class of PKC activators includes fatty esters derived from cyclopropanated MUFAs. In an embodiment, the cyclopropanated MUFA ester is selected from cyclopropanated elaidic acid methyl ester (shown below),

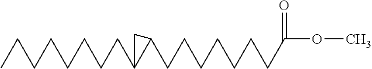 [SMILES: CCCCCCCCC1CC1CCCCCCCC(=O)OC]

and cyclopropanated oleic acid methyl ester (shown below).

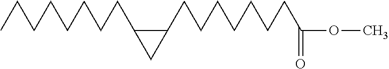 [SMILES: CCCCCCCCC1CC1CCCCCCCC(=O)OC]

Another class of PKC activators includes sulfates and phosphates derived from PUFAs, MUFAs, and their derivatives. In an embodiment, the sulfate is selected from DCPLA sulfate and DHA sulfate (CP6 form shown below).

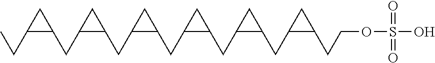 [SMILES: CCC1CC1CC1CC1CC1CC1CC1CC1CC1CC1CC1CC1CCOS(=O)(=O)O]

In one embodiment, the phosphate is selected from DCPLA phosphate and DHA phosphate (CP6 form shown below).

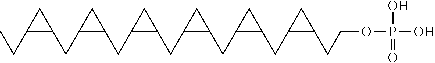 [SMILES: CCC1CC1CC1CC1CC1CC1CC1CC1CC1CC1CC1CC1CCOP(=O)(O)O]

In some embodiments, the PKC activator is selected from macrocyclic lactones, bryologs, diacylglycerols, isoprenoids, octylindolactam, gnidimacrin, ingenol, iripallidal, napthalenesulfonamides, diacylglycerol inhibitors, growth factors, polyunsaturated fatty acids, monounsaturated fatty acids, cyclopropanated polyunsaturated fatty acids, cyclopropanated monounsaturated fatty acids, fatty acids alcohols and derivatives, or fatty acid esters. In some embodiments, any two or more of the above disclosed PKC activators may be administered to the subject in combination.

In some embodiments, the PKC activator (e.g., bryostatin or bryolog) is administered in combination with one or more other substances (i.e., co-drugs, typically, non-PKC activating) known to promote remyelination or reduce neurological inflammation or the known symptoms of MS. Alternatively, the one or more co-drugs may favorably or synergistically augment the efficacy of the PKC activator in remyelinating neural tissue or treating neurological inflammation, particularly in MS. The one or more co-drugs may be administered separately (e.g., same or different days or weeks) but in tandem with the administration of the PKC activator, or the one or more co-drugs may be included within the same pharmaceutical formulation as the PKC activator, thereby being administered to the subject at the same time within the same dosage form. The co-drug may be, for example, an immunomodulating drug (e.g., glatiramer acetate), monoclonal antibody acting as α4 integrin antagonist (e.g., natalizumab), a hormone (e.g., melatonin), antibiotic (e.g., minocycline), steroid or corticosteroid (e.g., corticotrophin, cortisone, methylprednisolone, or clobetasol), cytokine in the interferon family (e.g., interferon beta-la or interferon beta 1-alpha), statin (e.g., simvastatin), or an antifungal or ERK activity enhancer (e.g., miconazole).

The PKC activators according to the present disclosure may be administered to a patient/subject in need thereof by conventional methods, such as oral, parenteral, transmucosal, intranasal, inhalation, or transdermal administration. Parenteral administration includes intravenous, intra-arteriolar, intramuscular, intradermal, subcutaneous, intraperitoneal, intraventricular, intrathecal, ICV, intracisternal injections or infusions and intracranial administration. A suitable route of administration may be chosen to permit crossing the blood-brain barrier. See e.g., J. Lipid Res. (2001) vol. 42, pp. 678-685, incorporated by reference herein.

The PKC activators can be compounded into a pharmaceutical composition suitable for administration to a subject using general principles of pharmaceutical compounding. In one aspect, the pharmaceutically acceptable formulation comprises a PKC activator and a pharmaceutically acceptable carrier. One or more co-drugs, as described above, may or may not also be included in the formulation.

The formulations of the compositions described herein may be prepared by any suitable method known in the art. In general, such preparatory methods include bringing at least one of the active ingredients into association with a carrier. If necessary or desirable, the resultant product can be shaped or packaged into a desired single- or multi-dose unit.

As discussed herein, carriers include, but are not limited to, one or more of the following: excipients; surface active agents; dispersing agents; inert diluents; granulating and disintegrating agents; binding agents; lubricating agents; sweetening agents; flavoring agents; coloring agents; preservatives; physiologically degradable compositions such as gelatin; aqueous vehicles and solvents; oily vehicles and solvents; suspending agents; dispersing or wetting agents; emulsifying agents, demulcents; buffers; salts; thickening agents; fillers; emulsifying agents; antioxidants; antibiotics; antifungal agents; stabilizing agents; and pharmaceutically acceptable polymeric or hydrophobic materials. Other additional ingredients that may be included in the compositions of the disclosure are generally known in the art and may be described, for example, in Remington's Pharmaceutical Sciences, Genaro, ed., Mack Publishing Co., Easton, Pa., 1985, and Remington's Pharmaceutical Sciences, 20^{th} Ed., Mack Publishing Co. 2000, both incorporated by reference herein.

In at least one embodiment, the carrier is an aqueous or hydrophilic carrier. In a further embodiment, the carrier may be water, saline, or dimethylsulfoxide. In another embodiment, the carrier is a hydrophobic carrier. Exemplary hydrophobic carriers include, for example, inclusion complexes, dispersions (such as micelles, microemulsions, and emulsions), and liposomes. See, e.g., Remington's: The Science and Practice of Pharmacy 20th ed., ed. Gennaro, Lippincott: Philadelphia, P A 2003, incorporated by reference herein. In addition, other compounds may be included either in the hydrophobic carrier or the solution, e.g., to stabilize the formulation.

In some embodiments, the compositions described herein may be formulated into oral dosage forms. For oral administration, the composition may be in the form of a tablet or capsule prepared by conventional means with, for example, carriers such as binding agents (e.g., pregelatinized maize starch, polyvinylpyrrolidone, or hydroxypropyl methylcellulose); fillers (e.g., lactose, microcrystalline cellulose, or calcium hydrogen phosphate); lubricants (e.g., magnesium stearate, talc, or silica); disintegrants (e.g., potato starch or sodium starch glycolate); or wetting agents (e.g., sodium lauryl sulfate). The tablets may be coated by methods generally known in the art.

In another embodiment, the compositions herein are formulated into a liquid preparation. Such preparations may be in the form of, for example, solutions, syrups or suspensions, or they may be presented as a dry product for constitution with water or other suitable vehicle before use. Such liquid preparations may be prepared by conventional means using pharmaceutically acceptable carriers, such as suspending agents (e.g., sorbitol syrup, cellulose derivatives, or hydrogenated edible fats); emulsifying agents (e.g., lecithin or acacia); non-aqueous vehicles (e.g., almond oil, oily esters, ethyl alcohol, or fractionated vegetable oils); and preservatives (e.g., methyl or propyl p-hydroxybenzoates, or sorbic acid). The preparations may also comprise buffer salts, flavoring, coloring, and sweetening agents as appropriate. In some embodiments, the liquid preparation is specifically designed for oral administration.

In another embodiment of the present disclosure, the compositions herein may be formulated for parenteral administration such as bolus injection or continuous infusion. Formulations for injection may be presented in unit dosage form, e.g., in ampoules, or in multi-dose containers, with an added preservative. The composition may be in the form of a suspension, solution, dispersion, or emulsion in oily or aqueous vehicles, and may contain a formulary agent, such as a suspending, stabilizing, and/or dispersing agent.

In another embodiment, the compositions herein may be formulated as depot preparations. Such formulations may be administered by implantation (for example, subcutaneously or intramuscularly) or by intramuscular injection. For example, the compositions may be formulated with a suitable polymeric or hydrophobic material (for example, as an emulsion in an acceptable oil) or ion exchange resin, or as a sparingly soluble derivative, for example, as a sparingly soluble salt.

In another embodiment, at least one PKC activator or combination thereof is delivered in a vesicle, such as a micelle, liposome, or an artificial low-density lipoprotein (LDL) particle. See, e.g., U.S. Pat. No. 7,682,627, the contents of which are herein incorporated by reference.

In some embodiments, at least one PKC activator or combination of PKC activators may be present in the pharmaceutical composition in an amount ranging from about 0.01% to about 100%, from about 0.1% to about 90%, from about 0.1% to about 60%, from about 0.1% to about 30% by weight, or from about 1% to about 10% by weight of the final formulation. In another embodiment, at least one PKC activator or combination of PKC activators may be present in the composition in an amount ranging from about 0.01% to about 100%, from about 0.1% to about 95%, from about 1% to about 90%, from about 5% to about 85%, from about 10% to about 80%, and from about 25% to about 75%.

The present disclosure further relates to kits that may be utilized for administering to a subject a PKC activator according to the present disclosure. The kits may comprise devices for storage and/or administration. For example, the kits may comprise syringe(s), needle(s), needle-less injection device(s), sterile pad(s), swab(s), vial(s), ampoule(s), cartridge(s), bottle(s), and the like. The storage and/or administration devices may be graduated to allow, for example, measuring volumes. In at least one embodiment, the kit comprises at least one PKC activator in a container separate from other components in the system.

The kits may also comprise one or more anesthetics, such as local anesthetics. In at least one embodiment, the anesthetics are in a ready-to-use formulation, for example, an injectable formulation (optionally in one or more pre-loaded syringes), or a formulation that may be applied topically. Topical formulations of anesthetics may be in the form of an anesthetic applied to a pad, swab, towelette, disposable napkin, cloth, patch, bandage, gauze, cotton ball, Q-Tip™, ointment, cream, gel, paste, liquid, or any other topically applied formulation. Anesthetics for use with the present disclosure may include, but are not limited to lidocaine, marcaine, cocaine, and xylocaine.

The kits may also contain instructions relating to the use of at least one PKC activator or a combination thereof. In another embodiment, the kit may contain instructions relating to procedures for mixing, diluting, or preparing formulations of at least one PKC activator or a combination thereof. The instructions may also contain directions for properly diluting a formulation of at least one PKC activator or a combination thereof in order to obtain a desired pH or range of pHs and/or a desired specific activity and/or protein concentration after mixing but prior to administration. The instructions may also contain dosing information. The instructions may also contain material directed to methods for selecting subjects for treatment with at least one PKC activator or a combination thereof.

The PKC activator can be formulated, alone in suitable dosage unit formulations containing conventional non-toxic pharmaceutically acceptable carriers, adjuvants and vehicles appropriate for each route of administration. Pharmaceutical compositions may further comprise other therapeutically active compounds which are approved for the treatment of neurodegenerative diseases or to reduce the risk of developing a neurodegenerative disorder.

All of the references, patents and printed publications mentioned in the instant disclosure are hereby incorporated by reference in their entirety into this application.

The following examples are provided by way of illustration to further describe certain preferred embodiments of the invention, and are not intended to be limiting of the present disclosure.

### EXAMPLES

In vitro or in vivo studies may be performed using a PKC activating compound, such as a bryostatin (e.g., bryostatin-1) or bryolog. An experimental autoimmune encephalomyelitis (EAE) mouse model, in particular, may be used for modeling demyelination or neurological inflammation, such as occurs in MS (A. P. Robinson et al., Hand. Clin. Neurol., 122, pp. 173-189, 2014). By known methods, EAE can be first initiated (induced) by immunization with a specific CNS antigen, such as myelin oligodendrocyte glycoprotein (MOG) or other inducer, to induce encephalomyelitis. Some of the mice are then administered the PKC activating compound either before, during, or after induction to determine the efficacy of the compound on initiation or development of EAE. In the case of administering the PKC activating compound before or at the start of MOG immunization, the PKC activating compound may result in prevention of the induction of EAE, which may indicate ability of the PKC activating compound to prevent demyelination, induce remyelination in later MS, or reduce or prevent neuroinflammation in early MS. In the case of administering the PKC activating compound after induction of EAE, the PKC activating compound may result in suppression, halting, or even reversal of EAE development, which may indicate ability of the PKC activating compound to suppress, halt, or even reverse demyelination (i.e., by remyelination) in MS.

Groups of 2-3 mice may be formed and housed in an approved research animal facility. Water may be given ad libitum. A first study involves three groups of mice with animals in each group dosed weekly for 1, 2, 3, or 6 consecutive weeks. Each group has its own control group containing the same number of mice. For example, mice in the first, second and third groups may receive an intravenous (i.v.) injection of 10 μg/m², 15 μg/m², and 25 μg/m² dose of a bryostatin, bryolog, or other PKC activating compound, respectively. For each dose, mice in that group may receive a single injection of a bryostatin, bryolog, or other PKC activating compound weekly for a predetermined number of consecutive weeks. Following dosing, mice are generally sacrificed and the blood and brain of each animal may be collected for further analysis.

In some cases, mice are dosed weekly with a bryostatin, bryolog, or other PKC activating compound at about 25 μg/m² for three consecutive weeks, followed by cessation of drug administration for three consecutive weeks, and then a second round of dosing at about 25 μg/m² for an additional three consecutive weeks (that is, a “3 on/3 off/3 on” dosing regimen). In other embodiments, mice are dosed at about 25 μg/m² at a “1 on/1 off” regimen for a total of nine weeks (e.g., one dose of a bryostatin, bryolog, or other PKC activating compound on weeks 1, 3, 5, 7, and 9, with no dosing in weeks 2, 4, 6, and 8). In other embodiments, mice are dosed at about 25 μg/m² for another regimen starting with “2 on/1 off” immediately followed by alternating “1 on/1 off” until reaching the ninth total week (i.e., one dose of a bryostatin, bryolog, or other PKC activating compound on weeks 1, 2, 4, 6, 8, with no dosing in weeks 3, 5, 7, and 9). Increasing the duration of the rest intervals (i.e., “off” intervals) to three weeks may significantly reduce PKC downregulation. That is, the “3 on/3 off” dosing regimen may increase brain PKC-ε levels in mice over the other regimens, thus resulting in particularly beneficial results.

Brain BDNF in mice may reach its highest level after three consecutive weekly doses of a bryostatin, bryolog, or other PKC activating compound at about 25 μg/m² and remain elevated after three additional consecutive weeks of no dosing, followed by three more consecutive weekly doses at about 25 μg/m². Since BDNF is a peptide that induces synaptogenesis (i.e., the formation of new synapses), a “3 on/3 off” regimen may maximize synaptogenesis and minimize PKC downregulation.

Further evaluation may be performed on a bryostatin, bryolog, or other PKC activating compound crossing the blood-brain-barrier (BBB) and the steady state levels of a bryostatin, bryolog, or other PKC activating compound in the brain and plasma of mice. In some embodiments, a bryostatin, bryolog, or other PKC activating compound administered intravenously crosses the BBB. In that case, the concentration of a bryostatin, bryolog, or other PKC activating compound in mice brain may be less than its concentration in plasma. However, the concentration in brain may be no less than two-fold lower than the plasma concentrations for comparable doses under steady-state conditions.

A weekly dosing regimen of a single injection of a bryostatin, bryolog, or other PKC activating compound at a dose of about 25 μg/m² for three consecutive weeks may be less effective at increasing the concentration of a bryostatin, bryolog, or other PKC activating compound in mice brain than a “1 on/1 off” or a “2 on/1 off” administration of the 25 μg/m² dose. In contrast, plasma concentrations of a bryostatin, bryolog, or other PKC activating compound may be greater when the drug is administered as a single injection for three consecutive weeks. Blood plasma concentrations of a bryostatin, bryolog, or other PKC activating compound may be less in mice receiving a 25 μg/m² dose as a “1 on/1 off” or a “2 on/1 off” administration. Without being bound to a specific theory, it may be hypothesized that the intermittent dosing regimen facilitates the transport of a bryostatin, bryolog, or other PKC activating compound across the BBB.

While there have been shown and described what are at present considered the preferred embodiments of the invention, those skilled in the art may make various changes and modifications which remain within the scope of the invention defined by the appended claims.

1. A method for treating multiple sclerosis in a subject in which demyelination of nerve cells has occurred, the method comprising administering to said subject a pharmaceutically effective amount of a PKC activator to result in remyelination of the nerve cells in said subject.

2. The method of claim 1, wherein the multiple sclerosis is advanced multiple sclerosis.

3. The method according to claim 1, wherein the method results in regeneration of lost synaptic connections in the brain of the subject.

4. The method according to claim 1, wherein the PKC activator is a macrocyclic lactone compound.

5. The method of claim 4, wherein the macrocyclic lactone compound is a bryostatin compound.

6. The method of claim 5, wherein the bryostatin compound is bryostatin-1.

7. The method of claim 4, wherein the macrocyclic lactone compound is a bryolog compound.

8. The method of claim 7, wherein the bryolog compound has any of the following structures: 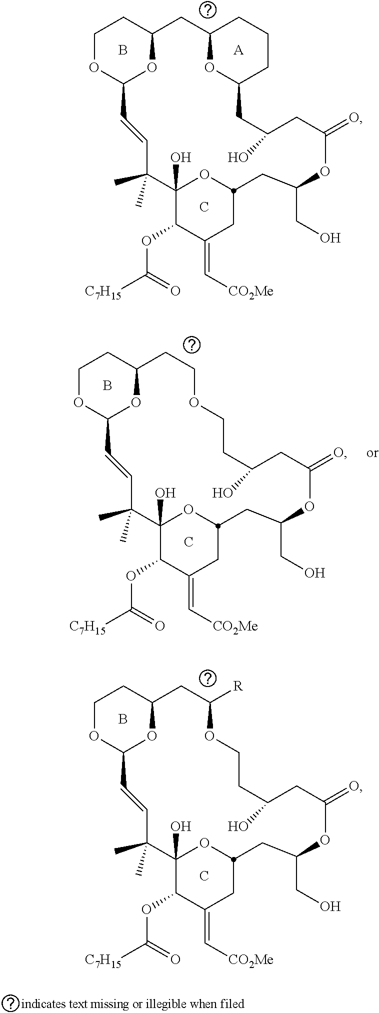
wherein R is selected from t-butyl, phenyl, and (CH₂)₃-p-Br-phenyl.

9. The method according to claim 1, wherein the PKC activator is a polyunsaturated fatty acid, ester thereof, cyclopropanated derivative thereof, epoxidized derivative thereof, or pharmaceutically acceptable salt thereof.

10. The method according to claim 1, wherein the PKC activator is a cyclopropanated polyunsaturated fatty acid ester having the following structure: 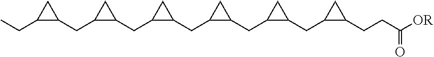
wherein R is an alkyl group.

11. The method according to claim 1, wherein the PKC activator is a growth factor or growth factor activating compound that functions as a PKC activator.

12. The method of claim 11, wherein the growth factor is selected from the group consisting of BDNF, HGF, NGF, and IGF.

13. The method according to claim 1, wherein the PKC activator is administered intravenously.

14. The method according to claim 1, wherein the PKC activator is administered as an oral dosage form.

15. The method according to claim 1, wherein the PKC activator is administered in an amount of 10-50 μg/m² weekly for at least 1 week.

16. The method according to claim 1, wherein the PKC activator is administered in an amount of 10-50 μg/m² weekly for at least 3 weeks.

17. The method according to claim 1, wherein the PKC activator is administered in an amount of 20-50 μg/m² weekly for at least 1 week.

18. The method according to claim 1, wherein the PKC activator is administered in an amount of 20-50 μg/m² weekly for at least 3 weeks.

19. The method according to claim 1, wherein the PKC activator is administered in an amount of 20-40 μg/m² weekly for at least 1 week.

20. The method according to claim 1, wherein the PKC activator is administered in an amount of 20-40 μg/m² weekly for at least 3 weeks.

21. The method according to claim 1, wherein the PKC activator is administered in an amount of 30-50 μg/m² weekly for at least 1 week.

22. The method according to claim 1, wherein the PKC activator is administered in an amount of 30-50 μg/m² weekly for at least 3 weeks.

23. The method according to claim 1, wherein the PKC activator is administered in an amount of 25-40 μg/m² weekly for at least 1 week.

24. The method according to claim 1, wherein the PKC activator is administered in an amount of 25-40 μg/m² weekly for at least 3 weeks.

25. The method according to claim 1, wherein the PKC activator is administered at an initial loading dose of about 15 micrograms per week for two consecutive weeks followed by about 12 micrograms on alternate weeks for a least four weeks.

26. The method according to claim 1, wherein the PKC activator is administered at an initial loading dose of about 24 micrograms per week for two consecutive weeks followed by about 20 micrograms on alternate weeks for a least four weeks.

27. The method according to claim 1, wherein the PKC activator is administered at an initial loading dose of about 48 micrograms per week for two consecutive weeks followed by about 40 micrograms on alternate weeks for a least four weeks.

28. The method according to claim 1, wherein the subject is not administered an NMDA receptor antagonist.

29. A method for reducing or preventing neuroinflammation in a subject having early multiple sclerosis or precursor symptoms to early MS, the method comprising administering to said subject a pharmaceutically effective amount of a PKC activator to result in reduction or prevention of neuroinflammation in said subject.

30. The method of claim 29, wherein the PKC activator is a macrocyclic lactone compound.

31. The method of claim 30, wherein the macrocyclic lactone compound is a bryostatin compound.

32. The method of claim 31, wherein the bryostatin compound is bryostatin-1.

33. The method of claim 30, wherein the macrocyclic lactone compound is a bryolog compound.

34. The method of claim 33, wherein the bryolog compound has any of the following structures: 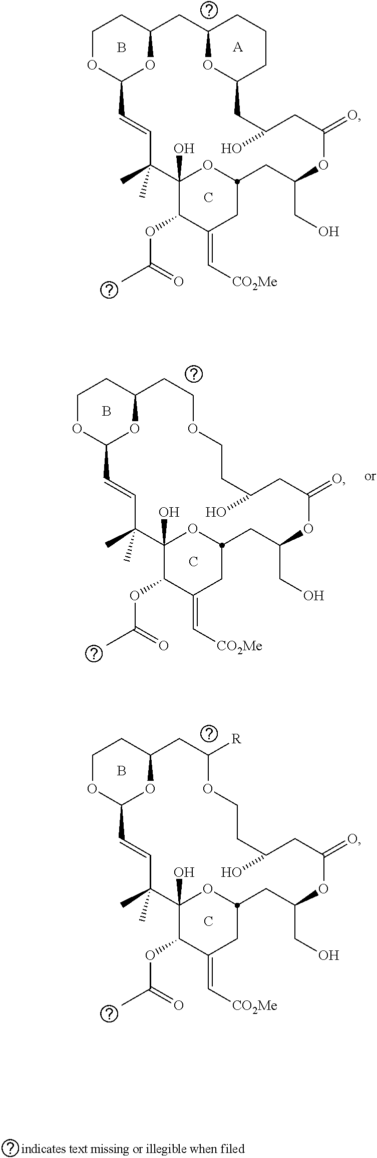
wherein R is selected from t-butyl, phenyl, and (CH₂)₃-p-Br-phenyl.

35. The method of claim 29, wherein the PKC activator is a polyunsaturated fatty acid, ester thereof, cyclopropanated derivative thereof, epoxidized derivative thereof, or pharmaceutically acceptable salt thereof.

36. The method of claim 29, wherein the PKC activator is a cyclopropanated polyunsaturated fatty acid ester having the following structure: 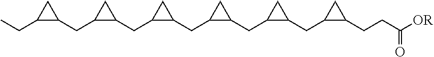
wherein R is an alkyl group.

37. The method of claim 29, wherein the PKC activator is a growth factor or growth factor activating compound that functions as a PKC activator.

38. The method of claim 37, wherein the growth factor is selected from the group consisting of BDNF, HGF, NGF, and IGF.

39. The method according to claim 29, wherein the subject is not administered an NMDA receptor antagonist.
